(19) United States
(12) Patent Application Publication
(10) Pub. No.: US 2025/0359735 A1
(43) Pub. Date: Nov. 27, 2025

(54) MEDICAL DEVICES WITH ELECTRONIC DRIVE ASSEMBLIES

(71) Applicant: Boston Scientific Scimed, Inc., Maple Grove, MN (US)

(72) Inventor: Jonathan MELCHOR, Providence, RI (US)

(73) Assignee: Boston Scientific Scimed, Inc., Maple Grove, MN (US)

(21) Appl. No.: 19/209,992

(22) Filed: May 16, 2025

Related U.S. Application Data
(60) Provisional application No. 63/649,973, filed on May 21, 2024.

(51) Int. Cl. A61B 1/005; A61B 1/00

(52) U.S. Cl. CPC A61B 1/0052; A61B 1/0004; A61B 1/00105; A61B 1/00124

(57) ABSTRACT

Medical devices, systems, and related methods useful for facilitating navigation through patient anatomy during a medical procedure are described. The medical device may include a handle including a proximal portion, a distal portion, and a middle portion between the proximal portion and the distal portion. The distal portion may be detachable from the middle portion by complementary mating elements. An articulation mechanism may be disposed within the distal portion of the handle, and configured to control articulation of a shaft coupled to the handle. A first electronic drive assembly may be configured to drive rotation of the middle portion and the distal portion relative to the proximal portion about a first axis. A second electronic drive assembly may be configured to drive rotation of the articulation mechanism about a second axis different from the first axis.

### CROSS-REFERENCE TO RELATED APPLICATIONS

This application claims the benefit of priority to U.S. Provisional Application No. 63/649,973, filed on May 21, 2024, which is incorporated by reference herein in its entirety.

### TECHNICAL FIELD

Aspects of the present disclosure relate generally to medical devices and actuation mechanisms for medical devices. More specifically, the present disclosure relates to medical systems and devices with electronic drive assemblies to facilitate users' grips on handles of the medical devices while articulating the medical devices.

### BACKGROUND

During a medical procedure, medical professionals often use medical devices during a procedure to navigate through patient anatomy using a steering mechanism to deflect a distal tip of the shaft of the medical device. However, steering mechanisms of current devices often require the user to rotate the handle of the medical device in an uncomfortable manner, leading to user fatigue and/or introducing risks to a patient during the procedure.

### SUMMARY

The present disclosure includes medical devices that facilitate steering a medical device in multiple planes. Each aspect disclosed herein may include one or more features described in connection with any other disclosed aspect.

According to some aspects of the present disclosure includes a medical device that includes a handle including a proximal portion, a distal portion, and a middle portion between the proximal portion and the distal portion. The distal portion may be detachable from the middle portion by complementary mating elements. An articulation mechanism may be disposed within the distal portion of the handle, and may be configured to control articulation of a shaft coupled to the handle. The medical device may include a first electronic drive assembly disposed within the proximal portion of the handle, and a second electronic drive assembly disposed within the middle portion of the handle. The first electronic drive assembly may be configured to drive rotation of the middle portion and the distal portion relative to the proximal portion about a first axis. The second electronic drive assembly may be configured to drive rotation of the articulation mechanism about a second axis different from the first axis.

According to some aspects, the proximal portion of the handle may include at least one actuator operably coupled to the first electronic drive assembly or the second electronic drive assembly. The at least one actuator may include a switch or a button. The at least one actuator may include a first actuator operably coupled to the first electronic drive assembly, and a second actuator operably coupled to the second electronic drive assembly. The first electronic drive assembly and the second electronic drive assembly may include a stepper motor. The second electronic drive assembly may be coupled to the articulation mechanism by a gear train. The gear train may include a worm gear and a spur gear. The articulation mechanism may include a cam having a plurality of teeth configured to engage the gear train. The articulation mechanism may include at least one articulation member fixed to the cam and configured to control articulation of the shaft. The complimentary mating elements may include at least one of a latch, a locking pin, a magnet, or a combination thereof. The medical device may include a position switch electrically coupled to the second electronic drive assembly, where the position switch is configured to limit rotation of the articulation mechanism relative to the second electronic drive assembly. The medical device may include the shaft, where a proximal end of the shaft is coupled to the distal portion of the handle, and where the articulation mechanism may include at least one articulation member extending from the distal portion of the handle through the shaft. One of the proximal portion or the middle portion of the handle may include a ridge that engages a groove of the other one of the proximal portion or the middle portion of the handle to permit rotation of the middle portion relative to the proximal portion. The proximal portion of the handle may include a circuit board, and the distal portion of the handle may include an electronic port to electronically couple the distal portion of the handle to the circuit board. The middle portion of the handle may include an electronic port to electronically couple the middle portion of the handle to the electronic port of the distal portion of the handle

The present disclosure also includes a medical device comprising a handle including a proximal portion, a distal portion, and a middle portion between the proximal portion and the distal portion. The distal portion may be detachable from the middle portion by complementary mating elements. An articulation mechanism may be configured to control articulation of a shaft coupled to the handle. The medical device may include a first electronic drive assembly including a stepper motor configured to drive rotation of the middle portion and the distal portion relative to the proximal portion about a longitudinal axis of the handle. The medical device may include a second electronic drive assembly including a stepper motor configured to drive rotation of the articulation mechanism about an axis transverse to the longitudinal axis.

According to some aspects, the handle may include a first actuator operably coupled to the stepper motor of the first electronic drive assembly and a second actuator operably coupled to the stepper motor of the second electronic drive assembly. The second electronic drive assembly may be coupled to the articulation mechanism by a gear train. The articulation mechanism may include a plurality of teeth configured to engage the gear train, and at least one articulation member configured to control articulation of the shaft. The medical device may include a position switch electrically coupled to the stepper motor of the second electronic drive assembly, where the position switch is configured to limit rotation of the articulation mechanism relative to the second electronic drive assembly.

The present disclosure also includes a medical device comprising a handle including a proximal portion, a distal portion, and a middle portion between the proximal portion and the distal portion; a first electronic drive assembly configured to drive rotation of the middle portion and the distal portion relative to the proximal portion about a longitudinal axis of the handle; an articulation mechanism configured to control articulation of a shaft coupled to the handle; a second electronic drive assembly configured to drive rotation of the articulation mechanism about an axis transverse to the longitudinal axis; and a position switch electrically coupled to the second electronic drive assembly. The position switch may be configured to limit rotation of the articulation mechanism relative to the second electronic drive assembly.

### BRIEF DESCRIPTION OF THE FIGURES

The accompanying drawings, which are incorporated in and constitute a part of this application, illustrate aspects of this disclosure and together with the description, serve to explain the principles of this disclosure.

FIG. 1 depicts an exemplary medical device according to some aspects of the present disclosure.

FIG. 2 depicts a cross-sectional side view of the handle according to some aspects of the present disclosure.

FIG. 3 depicts a perspective view of the proximal portion of the handle of FIG. 2.

FIG. 4 depicts a back perspective view of the distal portion of the handle of FIG. 2.

FIGS. 5-8 depict complementary mating elements of the handle of FIG. 2.

FIG. 9 depicts a front perspective cross-sectional view of features of the handle of FIG. 2.

FIG. 10 depicts a front perspective cut-away view of the distal portion of the handle of FIG. 2.

FIGS. 11 and 12 depict complementary mating elements of the handle of FIG. 2.

### DETAILED DESCRIPTION

Both the foregoing general description and the following detailed description are exemplary and explanatory only and are not restrictive of the features, as claimed. As used herein, the terms “comprises,” “comprising,” “including,” “includes,” “having,” “has,” or any other variation thereof, are intended to cover a non-exclusive inclusion, such that a process, method, article, or apparatus that comprises a list of elements does not include only those elements, but may include other elements not expressly listed or inherent to such process, method, article, or apparatus. The term “exemplary” is used in the sense of “example,” rather than “ideal.” Relative terms such as “about,” “substantially,” and “approximately,” etc., are used to indicate a possible variation of ±10% of the stated numeric value or range. The terms “proximal” and “distal” are used herein to refer to the relative positions of the components of exemplary medical devices. As used herein, “proximal” refers to a position relatively closer to the exterior of the body or closer to an operator using the medical device (see proximal “P” and distal “D” directional arrows in the figures). In contrast, “distal” refers to a position relatively further away from the operator using the medical device, or closer to the interior of the body

Aspects of the present disclosure are now described with reference to exemplary medical devices, systems, and methods useful for facilitating navigation through patient anatomy during a medical procedure. For instance, exemplary medical devices herein may include a handle configured to rotate a shaft of the medical device without rotating the entire handle and/or configured to deflect a distal tip of the shaft, e.g., via one or more electronic drive assemblies. The electronic drive assembly(ies) may receive user input to one or more actuators of the handle.

Reference will now be made in detail to examples to help illustrate aspects of the present disclosure through the accompanying drawings. Wherever possible, the same reference numbers will be used throughout the drawings to refer to the same or like parts.

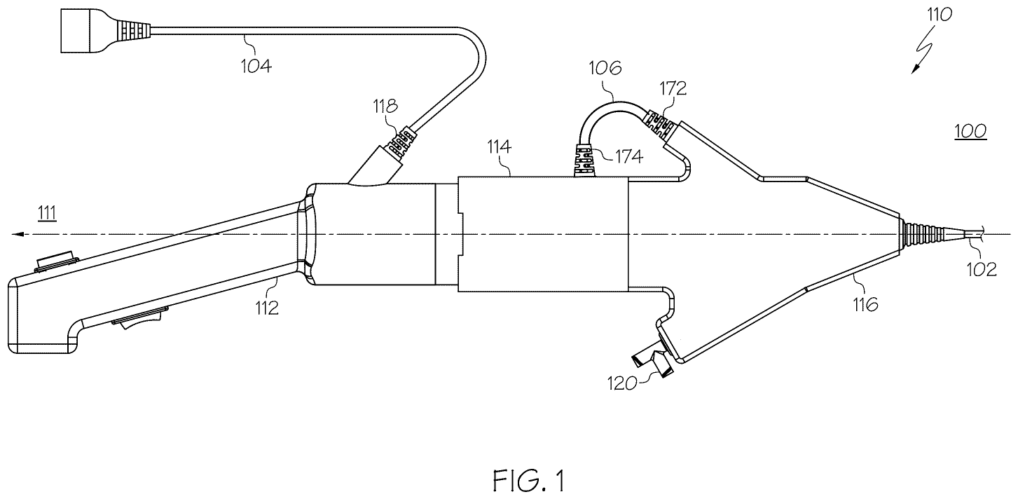

FIG. 1 illustrates an exemplary medical device 100 including a handle 110 and a shaft 102. The medical device 100 may be any suitable device configured to allow a user (e.g., medical professional) to access internal areas of a subject's body, e.g., to perform medical diagnoses and/or treatments on the subject. For example, the medical device 100 may be a ureteroscope, an endoscope, a hysteroscope, a bronchoscope, a cystoscope, or other scope or similar medical device.

As shown and discussed herein, the handle 110 includes a proximal portion 112, a distal portion 116, and a middle portion 114 between the proximal portion 112 and the distal portion 116. In some examples, the distal portion 116 may be selectively detachable from the middle portion 114, e.g., facilitating re-use of the proximal portion 112 and middle portion 114.

The handle 110 may include an umbilicus cord 104 and/or at least one port 118 configured to be coupled with the umbilicus cord 104. The umbilicus cord 104 may be used provide power transmission to medical device 100 (e.g., via an external power source) and/or data transmission to and from medical device 100, such as image data from an optical sensor and/or pressure data from a pressure sensor during operation of the medical device 100 and/or auxiliary instruments. The port 118 is shown on the proximal portion 112 of the handle 110 in this example but may be provided on other portions of handle 110. The handle 110 may include an umbilicus cord 106 and/or at least one port 172, 174 configured to be coupled with the umbilicus cord 106. The umbilicus cord 106 may be used to electrically couple the middle portion 114 and the distal portion 116 of the handle 110. For example, the umbilicus cord 106 may be used to provide data transmission between the middle portion 114 and the distal portion 116 of the handle 110, such as position data from an electronic drive assembly in the middle portion 114 and/or position data from an articulation mechanism in the distal portion 116.

Handle 110 also may include one or more ports in communication with one or more working channels of the shaft 102. For example, the handle 110 may include a port 120 in communication with a working channel defined by the shaft 102. Auxiliary instruments, such as, for example, biopsy forceps, graspers, baskets, snares, and/or other devices may be inserted into the port 120 and advanced through the working channel to exit through a distal opening of the shaft 102 into a subject's body to perform a medical procedure. Additionally or alternatively, the port 120 may be used to provide fluid and/or suction to the working channel during a medical procedure. The port 120 is shown on the distal portion 116 of the handle 110 in this example but may be provided on other portions of handle 110.

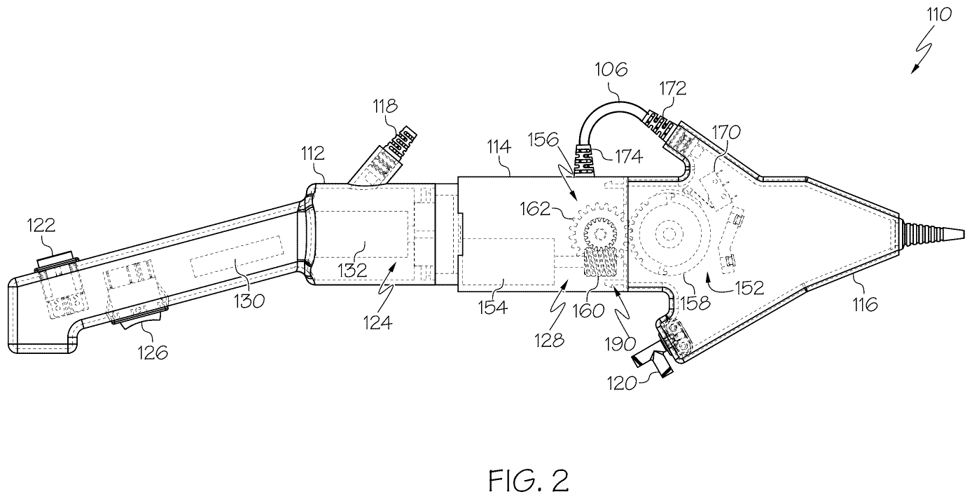

FIG. 2 illustrates a cross-sectional view of the handle 110 according to some aspects of the disclosure. The handle 110 includes one or more actuators configured to receive user input to drive one or more corresponding electronic drive assemblies. The electronic drive assembly(ies) may control rotation of the middle portion 114 and distal portion 116 relative to the proximal portion 112 about an axis substantially parallel to a longitudinal axis of the handle 110, e.g., shown as a first axis 111 in FIG. 1.

For example, the handle 110 includes a first actuator 122 operably coupled to a first electronic drive assembly 124. Actuation of the first actuator 122 drives rotation of the first electronic drive assembly 124, which in turn rotates the middle portion 114 and the distal portion 116 relative to the proximal portion 112 about the first axis 111. The handle 110 may further include a second actuator 126 operably coupled to a second electronic drive assembly 128. Actuation of the second actuator 126 drives rotation of the second electronic drive assembly 128, which in turn drives an articulation mechanism 152 of the handle 110 to deflect the distal end of the shaft 102.

While FIG. 2 illustrates an example with two actuators 122, 126, in some aspects of the present disclosure, the handle 110 includes one actuator coupled to both the first and second electronic drive assemblies 124, 128. Further, the present disclosure includes medical devices that include more than two actuators to control the first and second electronic drive assemblies 124, 128.

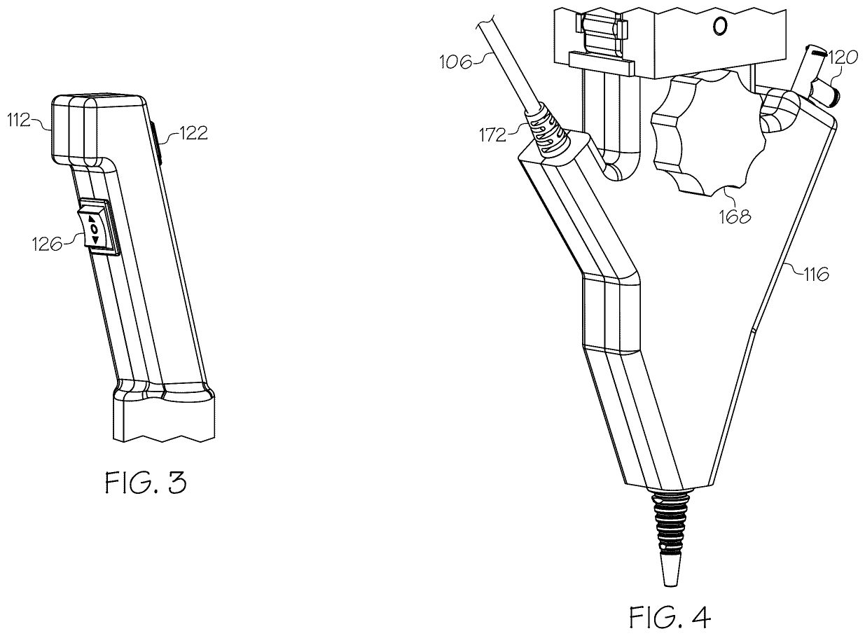

The handle 110 may have any suitable shape able to be gripped by a user's hand, e.g., a grip portion disposed on the proximal portion 112 and/or middle portion 114. As shown in the example illustrated in FIGS. 2 and 3, the proximal portion 112 of the handle 110 includes the first actuator 122 and the second actuator 126. The first actuator 122 and/or the second actuator 126 may include, e.g., a button, switch (e.g., toggle switch), user interface, etc. In at least one example, each of the first actuator 122 and second actuator 126 includes a double pole, double throw (DPDT) rocker switch to control clockwise and counterclockwise rotations associated with the first and second electronic drive assemblies 124, 128. In other examples, the handle 110 may include one actuator configured to provide for four-way movements to control clockwise and counterclockwise rotations associated with the first and second electronic drive assemblies 124, 128 (e.g., up/down to control the first electronic drive assembly 124 and left/right to control the second electronic drive assembly 128, or vice versa). In other examples, the handle 110 may include four actuators, two actuators to control respective clockwise and counterclockwise rotations associated with the first electronic drive assembly 124, and two actuators to control respective clockwise and counterclockwise rotations associated with the first electronic drive assembly 128,

The handle 110 may further include at least one circuit board 130 to control various electrical components of the medical device 100. The circuit board 130 may include program instructions stored on computer-readable memory and configured to control operation of one or more electronic components of the medical device 100. As shown in FIG. 2, the circuit board 130 is disposed in the proximal portion 112 of the handle 110 although this is exemplary and non-limiting of other examples. The circuit board 130 is electrically coupled to the first actuator 122 and second actuator 126 and to the first electronic drive assembly 124 and the second electronic drive assembly 128. In response to user input via the first actuator 122 and/or the second actuator 126, the circuit board 130 may transmit electrical signals corresponding to operation instructions for the first electronic drive assembly 124 and/or the second electronic drive assembly 128.

In some examples, the first electronic drive assembly 124 is configured to drive rotation of the middle portion 114 and the distal portion 116 of the handle 110 relative to the proximal portion 112. The first electronic drive assembly 124 may include a stepper motor 132. For example, the stepper motor 132 may be disposed within the proximal portion 112 and/or the middle portion 114 of the handle 110. The stepper motor 132 may be configured to rotate about the first axis 111 (e.g., clockwise and/or counter-clockwise) in response to actuation of the first actuator 122. Rotating the stepper motor 132 about the first axis 111 may cause the middle portion 114 of the handle 110 to rotate relative to the proximal portion 112.

The middle portion 114 of the handle 110 may be coupled to the distal portion 116 and/or to the proximal portion 112 via complementary mating elements. In some examples, the mating elements may provide for portions of the handle 110 to be selectively detachable, e.g., to assemble and disassemble the handle 110. In the example illustrated in the figures, the distal portion 116 is coupled to the middle portion 114 via complementary mating elements 176 (discussed further below), and the proximal portion 112 is coupled to the middle portion 114 via complementary mating elements 190. Complementary mating elements suitable for the present disclosure may include, but are not limited to snap-fit, grooves, projections, threads, latches, locking pins, magnets, and friction fit.

When assembled (when the distal portion 116 is coupled to the middle portion 114, the middle portion also being coupled to the proximal portion 112), the middle portion 114 and the distal portion 116 may be configured to rotate together relative to the proximal portion 112 when the first electronic drive assembly is engaged. The first electronic drive assembly 124 may drive rotation in a first direction (e.g., clockwise) and/or a second direction opposite the first direction (e.g., counter-clockwise). In some examples, the first electronic drive assembly 124 may be capable of rotating the middle portion 114 and the distal portion 116 a full 360 degrees about the first axis 111. In other examples, the handle 110 may include an internal stop that limits rotation, e.g., such that rotation of the middle portion 114 and/or the distal portion 116 relative to the proximal portion 112 is less than 360 degrees.

Complementary mating elements 190 are configured to secure the proximal portion 112 to the middle portion 114. In some examples herein, the mating elements 190 allow for relative rotation between the proximal portion 112 and the middle portion 114 without detaching the proximal portion 112 from the middle portion 114. The distal end of the proximal portion 112 of the handle 110 may be shaped and dimensioned to complementarily engage the proximal end of the middle portion 114. In some examples, at least one of the proximal portion 112 or middle portion 114 may include one or more projections or ridges that engage one or more complementary depressions or grooves of the other one of the proximal portion 112 or middle portion 114.

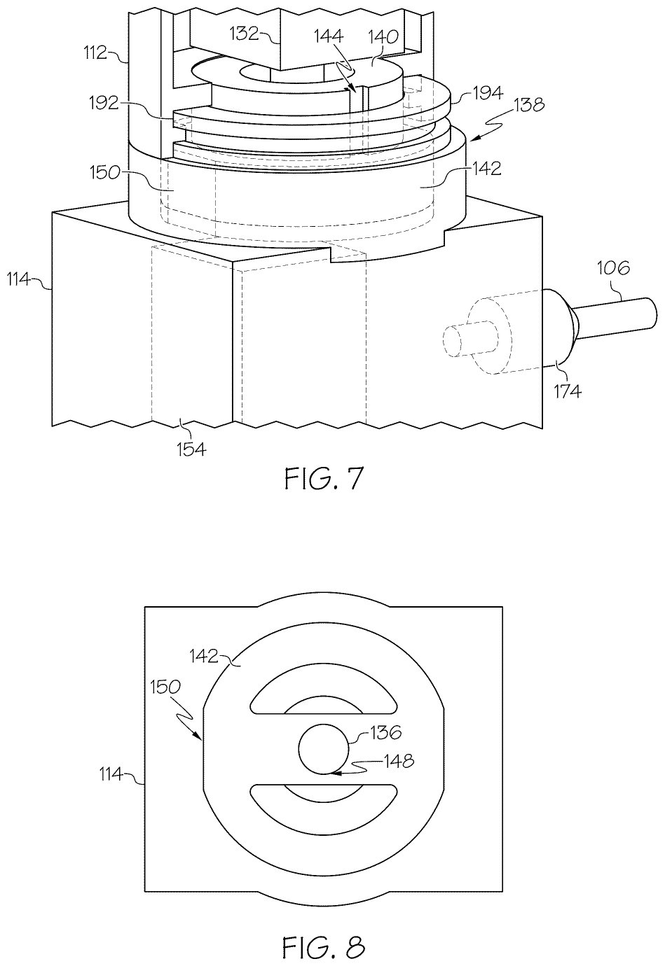

As shown in FIGS. 5-7, for example, the proximal portion 112 may include a ridge 192 (FIG. 5) configured to engage a groove 194 (FIG. 6) of the middle portion 114. As shown in FIG. 7, when the handle 110 is assembled, the ridge 192 is disposed in the groove 194 to thereby secure the proximal portion 112 to the middle portion 114. Engagement between the ridge 192 and the groove 194 longitudinally secures the proximal portion 112 to the middle portion 114 (e.g., at respective positions along the first axis 111), yet allows rotation of the middle portion 114 relative to the proximal portion 112. In other words, the complementary mating elements 190 allow the middle portion 114 to rotate relative to the proximal portion 112 and prevent translation of the middle portion 114 relative to the proximal portion 112.

As illustrated in FIG. 7, the stepper motor 132 of the first electronic derive assembly 124 may be coupled to a drive shaft 136 extending distally, e.g., along the first axis 111. The drive shaft 136 extends distally through a connector 138, e.g., at least partially disposed in the middle portion 114 of the handle 110. The connector 138 may define a channel extending longitudinally from a proximal portion 140 to a distal portion 142 of the connector 138. For example, the proximal portion 140 may be disposed in the proximal portion 112 of the handle 110, and the distal portion 142 may be disposed in the middle portion 114 of the handle 110. The channel of the connector 138 may be shaped and dimensioned to provide for an electrical connection between the proximal portion 112 and the middle portion 114 (e.g., via one or more wires) and to receive the drive shaft 136. The proximal portion 140 of the connector 138 may be secured to a proximal end of the drive shaft 136. The proximal portion 140 may include a slot 144 configured to engage a protrusion 146 of the proximal portion 112 of the handle 110, e.g., along an inner surface of the proximal portion 112. Engagement between the protrusion 146 and the slot 144 may rotationally fix the proximal portion 140 of the connector 138 relative to the proximal portion 112 (e.g., prevents rotation of the proximal portion 140 of the connector 138 relative to the proximal portion 112 about the first axis 111). Furthermore, the distal portion 142 of the connector 138 may be secured to a distal end of the drive shaft 136. The drive shaft 136 includes a keyed surface 148 (e.g., D-shape in this example, although other configurations are contemplated) configured to engage a complementary opening formed in the distal portion 142, such that rotating the drive shaft 136 (e.g., via the first electronic drive assembly 124) drives rotation of the distal portion 142 (e.g., about the first axis 111). The distal portion 142 includes a keyed outer surface 150 configured to engage an inner surface of the middle portion 114 of the handle 110 having a complementary shape, such that rotating the distal portion 142 of the connector 138 drives rotation of the middle portion 114 relative to the proximal portion 112 (e.g., due to engagement with the keyed outer surface 150).

As discussed above, the second electronic drive assembly 128 may be configured to control the articulation mechanism 152 to deflect the distal end of the shaft 102. Referring to FIG. 2, the second electronic drive assembly 128 is shown in this example disposed in the middle portion 114 and operably coupled to the distal portion 116 of the handle 110. The second electronic drive assembly 128 may include a stepper motor 154. For example, the stepper motor 154 may be configured to rotate clockwise and/or counter-clockwise (e.g., about an axis transverse to the longitudinal axis of the handle 110, second axis 115) in response to the second actuator 126 (e.g., engaged by user input). Rotating the stepper motor 154 may drive a gear train 156, which in turn controls the articulation mechanism 152. For example, the second actuator 126 may have a first position corresponding to rotation of the stepper motor 154 in a first direction (e.g., clockwise) and a second position corresponding to rotation of the stepper motor 154 in a second direction (e.g., counter-clockwise). Rotation of the stepper motor 154 in the first and second directions may correspond to deflecting the distal end of the shaft 102 in opposite directions along a plane. The gear train 156 may be operably coupled between the second electronic drive assembly 128 and the articulation mechanism 152. The gear train 156 may include a plurality of gears operably coupling the stepper motor 154 of the second electronic drive assembly 128 and a cam 158 of the articulation mechanism 152.

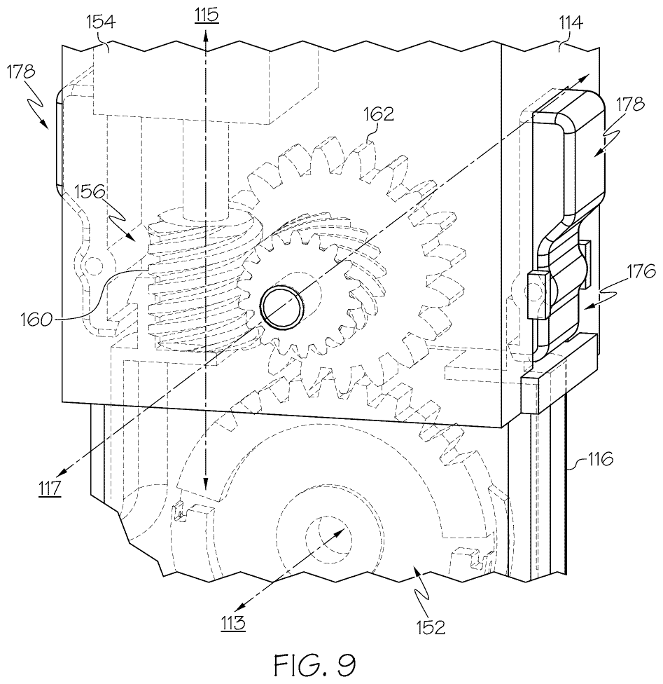

FIG. 9 illustrates a cross-sectional perspective view of the gear train 156 according to some aspects of the present disclosure. The gear train 156 may include a worm gear 160 operably coupled to the stepper motor 154, such that rotating the stepper motor 154 drives rotation of the worm gear 160 about the second axis 115. The second axis 115 may be parallel to the first axis 111 and parallel to the longitudinal axis of the handle 110. The gear train 156 may include a spur gear 162 operably coupled to the worm gear 160, such that rotation of the worm gear 160 drives rotation of the spur gear 162 clockwise and/or counter-clockwise (e.g., about an axis transverse to the longitudinal axis of the handle 110, e.g., third axis 117). The third axis 117 may be parallel to a fourth axis 113. As further shown in FIG. 9, the worm gear 160 includes a plurality of teeth configured for complementary engagement with a plurality of teeth of the spur gear 162. In some aspects, the worm gear 160 and the spur gear 162 may have a gear ratio which may be adjusted. The gear train 156 may additionally or alternatively include one or more other gears operably coupling the second electronic drive assembly 128 and the articulation mechanism 152. The spur gear 162 may be at or proximate the distal end of the middle portion 114 of the handle 110, such that one or more teeth of the spur gear 162 are available for complementary engagement with one or more teeth of the articulation mechanism 152 at or proximate the proximal end of the distal portion 116 of the handle 110. Actuating the second actuator 126 may cause the stepper motor 154 to rotate about the axis 115, which in turn may drive rotation of the worm gear 160. Further, rotation of the worm gear 160 may drive rotation of the spur gear 162, and which in turn may drive rotation of the cam 158 of the articulation mechanism 152.

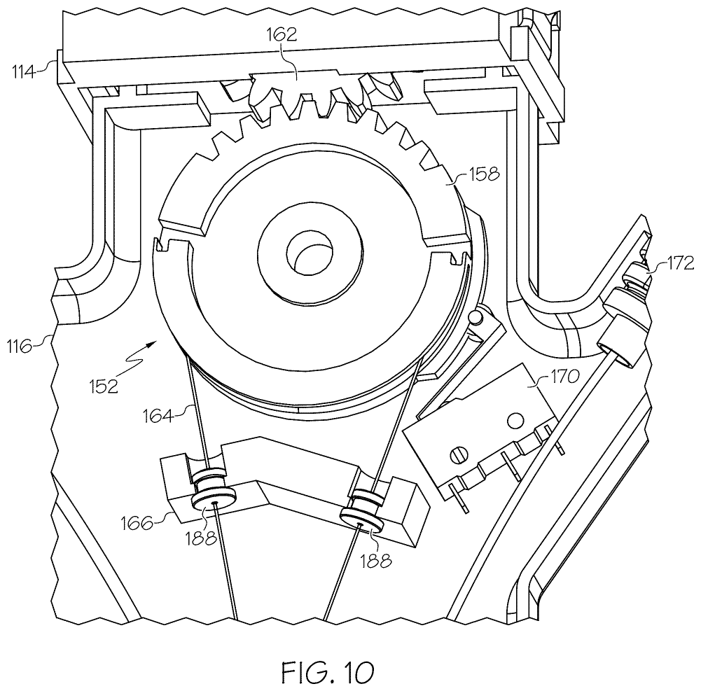

FIG. 10 illustrates a perspective cut-away view of the articulation mechanism 152 according to some aspects of the present disclosure. As shown, the cam 158 of the articulation mechanism 152 is configured to be operably coupled to the gear train 156. For example, the cam 158 may include one or more teeth configured for complementary engagement with the one or more teeth of the spur gear 162. As discussed above, rotation of the second electronic drive assembly 128 may drive the gear train 156 and rotation of the cam 158 about the fourth axis 113. The articulation mechanism 152 further includes at least one articulation member 164 (e.g., pull wire) extending from the distal portion 116 of the handle 110 and through the shaft 102 for deflecting the distal end of the shaft 102 relative to the handle 110. As shown in FIG. 10, the at least one articulation member 164 is operably coupled to the cam 158, e.g., fixed to the cam 158 by a fixation portion 166. The handle 110 may include one or more ferrules 188, e.g., to facilitate insertion and/or operation of the articulation member(s) 164. For example, the ferrules 188 may stop or restrict movement of the articulation member(s) 164 received therein.

Rotation of the handle 110 may be independent of deflection of the shaft 102. For example, once a user has provided input (e.g., to the second actuator) to operate the articulation mechanism 152 as described above, such that the shaft 102 is in a desired position (e.g., deflected away from the longitudinal axis of the medical device 100), the user may adjust the plane in which the deflected shaft 102 extends by rotating the shaft 102. The user may actuate the first actuator 122 to rotate the distal portion 116 of the handle 110, which thereby rotates the shaft 102 (and rotates the middle portion 114 of the handle 110 as discussed above). Since the middle portion 114 and the distal portion 116 are rotatable together relative to the proximal portion 112 of the handle 110, the user need not adjust the grip on the proximal portion 112 to rotate the shaft 102.

Optionally, the handle 110 may include a mechanism to limit deflection of the shaft 102. For example, the handle 110 may include a position switch 170 electrically coupled to the second electronic drive assembly 128. The position switch 170 may transmit electrical signals corresponding to one or more parameters of the articulation mechanism 152. For example, the position switch 170 may transmit electrical signals corresponding to a relative position of the cam 158 relative to the second electronic drive assembly 128. The position switch 170 may be operably coupled to the cam 158, such that rotating the cam 158 relative to the second electronic drive assembly 128 causes engagement and/or disengagement between the cam 158 and the position switch 170. This engagement may allow the position switch 170 to determine a position of the articulation mechanism 152 relative to the second electronic drive assembly 128. In response to signals from the position switch 170, the second electronic drive assembly 128 may be configured to adjust rotation of the articulation mechanism 152 (e.g., stop or limit rotation). For example, the stepper motor 154 may stop rotating to thereby stop deflecting the shaft 102 relative to the handle 110.

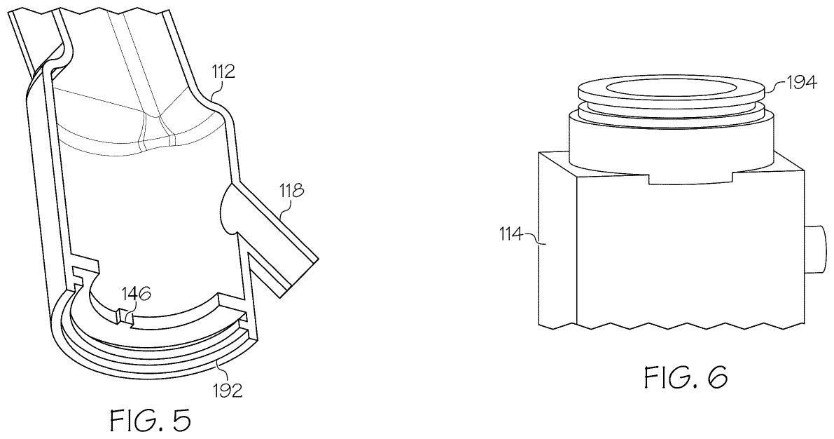

Optionally, the articulation mechanism 152 may include an actuator that permits manual control over the articulation mechanism 152. For example, FIG. 4 illustrates the distal portion 116 of the handle 110 with a knob 168 operably coupled to the articulation mechanism 152. The knob 168 may have any shape and dimension configured to be grasped by the user. For example, the knob 168 may include surface features, e.g., annular grooves, disposed thereon to facilitate being gripped by the user's hand. The knob 168 may be operably coupled to the articulation mechanism 152 by an elongate member extending through, or otherwise rotatably coupled to, the cam 158 of the articulation mechanism 152. Rotating the knob 168 may cause a corresponding rotation of the cam 158 to control movement of the articulation member(s) 164 to deflect the shaft 102 along a plane as discussed above.

The handle 110 may include features to permit electrical or electronic connection between the proximal portion 112, middle portion 114 and/or distal portion 116 of the handle 110 when the handle 110 is assembled. For example, the distal portion 116 of the handle 110 may include an electronic port 172 configured for electrically coupling the distal portion 116 to the proximal portion 112 and/or the middle portion 114 of the handle 110, e.g., via a corresponding electronic port 174 of the middle portion 114. The handle 110 may include features to provide power between the proximal portion 112, middle portion 114 and/or distal portion 116 of the handle 110. For example, the port 118 of the proximal portion 112 may be electrically coupled to an external power source, and electrically coupled to the middle portion 114 and/or distal portion 116 via one or more electrical connections (e.g., wires) extending from the port 118 through the connector 138.

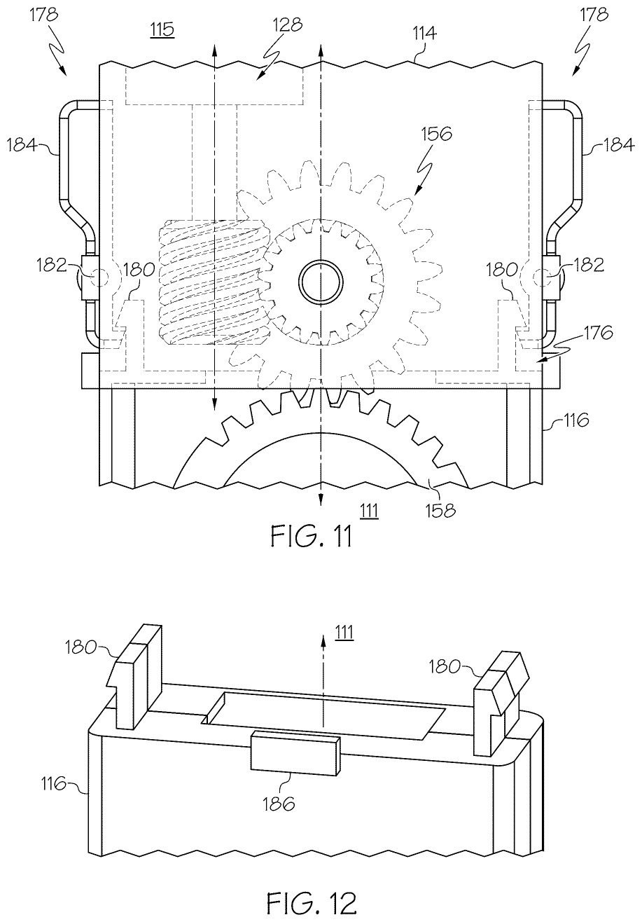

As discussed above, the distal portion 116 may be detachable from the middle portion 114 of the handle 110, e.g., via complementary mating elements 176. In this example, the complementary mating elements 176 include a lock 178 disposed on the middle portion 114 (FIG. 11), and a retention member 180 extending proximally from the distal portion 116 (FIG. 12). The lock 178 may include a first spring lock and a second spring lock disposed on opposing sides of the middle portion 114. The retention member 180 may include a plurality of members extending proximally from the distal portion 116 for engagement with the spring locks. A proximal end of the retention member 180 may be engaged with a distal end of the lock 178, which is shaped and dimensioned to deflect the retention member 180 radially inward. The lock 178 may include one or more compression members 182 (e.g., one or more springs) configured to apply compressive force to maintain engagement with the retention member 180. The lock 178 may include at least on actuator 184 disposed on the middle portion 114, and operably coupled to the one or more compression members 182. For instance, as shown in FIG. 11, the actuator(s) 184 include a pair of depressible configured to translate radially inward relative to release compressive force applied by the compression members 182 to disengage the retention member 180.

The complementary mating elements 176 may further include a protrusion 186 disposed on the distal portion 116 of the handle 110 configured to engage a complementary groove or slot of the middle portion 114. The protrusion 186 may facilitate orientation of the distal portion 116 relative to the middle portion 114 while assembling the handle 110.

In some aspects, the distal portion 116 is a detachable, single-use device. Since the distal portion 116 is detachable, the proximal portion 112 and middle portion 114 of the handle 110 may be reusable between medical procedures. For instance, after performing a first medical procedure the distal portion 116 may be discarded, and thereby allowing the proximal portion 112 and middle portion 114 to attach to another distal portion 116 to perform a second medical procedure.

While principles of this disclosure are described herein with reference to illustrative examples, it should be understood that the disclosure is not limited thereto. Those having ordinary skill in the art and access to the teachings provided herein will recognize additional modifications, embodiments, and substitution of equivalents all fall within the scope of the features described herein. Accordingly, the claimed features are not to be considered as limited by the foregoing description.

What is claimed is:

1. A medical device, comprising:
a handle including a proximal portion, a distal portion, and a middle portion between the proximal portion and the distal portion, wherein the distal portion is detachable from the middle portion by complementary mating elements;
an articulation mechanism disposed within the distal portion of the handle, wherein the articulation mechanism is configured to control articulation of a shaft coupled to the handle;
a first electronic drive assembly disposed within the proximal portion of the handle, wherein the first electronic drive assembly is configured to drive rotation of the middle portion and the distal portion relative to the proximal portion about a first axis; and
a second electronic drive assembly disposed within the middle portion of the handle, wherein the second electronic drive assembly is configured to drive rotation of the articulation mechanism about a second axis different from the first axis.

2. The medical device of claim 1, wherein the proximal portion of the handle includes at least one actuator operably coupled to the first electronic drive assembly or the second electronic drive assembly.

3. The medical device of claim 2, wherein the at least one actuator includes a switch or a button.

4. The medical device of claim 2, wherein the at least one actuator includes a first actuator operably coupled to the first electronic drive assembly, and a second actuator operably coupled to the second electronic drive assembly.

5. The medical device of claim 1, wherein each of the first electronic drive assembly and the second electronic drive assembly includes a stepper motor.

6. The medical device of claim 1, wherein the second electronic drive assembly is coupled to the articulation mechanism by a gear train.

7. The medical device of claim 6, wherein the gear train includes a worm gear and a spur gear.

8. The medical device of claim 6, wherein the articulation mechanism includes a cam having a plurality of teeth configured to engage the gear train.

9. The medical device of claim 8, wherein the articulation mechanism includes at least one articulation member fixed to the cam and configured to control articulation of the shaft.

10. The medical device of claim 1, wherein the complimentary mating elements include at least one of a latch, a locking pin, a magnet, or a combination thereof.

11. The medical device of claim 1, further comprising a position switch electrically coupled to the second electronic drive assembly, wherein the position switch is configured to limit rotation of the articulation mechanism relative to the second electronic drive assembly.

12. The medical device of claim 1, further comprising the shaft, wherein a proximal end of the shaft is coupled to the distal portion of the handle, and wherein the articulation mechanism includes at least one articulation member extending from the distal portion of the handle through the shaft.

13. The medical device of claim 1, wherein one of the proximal portion or the middle portion of the handle includes a ridge that engages a groove of the other one of the proximal portion or the middle portion of the handle to permit rotation of the middle portion relative to the proximal portion.

14. The medical device of claim 1, wherein the proximal portion of the handle includes a circuit board, and wherein the distal portion of the handle includes an electronic port to electronically couple the distal portion of the handle to the circuit board.

15. The medical device of claim 14, wherein the middle portion of the handle includes an electronic port to electronically couple the middle portion of the handle to the electronic port of the distal portion of the handle.

16. A medical device, comprising:
a handle including a proximal portion, a distal portion, and a middle portion between the proximal portion and the distal portion, wherein the distal portion is detachable from the middle portion by complementary mating elements;
an articulation mechanism configured to control articulation of a shaft coupled to the handle;
a first electronic drive assembly including a stepper motor configured to drive rotation of the middle portion and the distal portion relative to the proximal portion about a longitudinal axis of the handle; and
a second electronic drive assembly including a stepper motor configured to drive rotation of the articulation mechanism about an axis transverse to the longitudinal axis.

17. The medical device of claim 16, wherein the handle includes a first actuator operably coupled to the stepper motor of the first electronic drive assembly and a second actuator operably coupled to the stepper motor of the second electronic drive assembly.

18. The medical device of claim 16, wherein the second electronic drive assembly is coupled to the articulation mechanism by a gear train, and wherein the articulation mechanism includes a plurality of teeth configured to engage the gear train, and at least one articulation member configured to control articulation of the shaft.

19. The medical device of claim 15, further comprising a position switch electrically coupled to the stepper motor of the second electronic drive assembly, wherein the position switch is configured to limit rotation of the articulation mechanism relative to the second electronic drive assembly.

20. A medical device, comprising:
a handle including a proximal portion, a distal portion, and a middle portion between the proximal portion and the distal portion;
a first electronic drive assembly configured to drive rotation of the middle portion and the distal portion relative to the proximal portion about a longitudinal axis of the handle;
an articulation mechanism configured to control articulation of a shaft coupled to the handle;
a second electronic drive assembly configured to drive rotation of the articulation mechanism about an axis transverse to the longitudinal axis; and
a position switch electrically coupled to the second electronic drive assembly, wherein the position switch is configured to limit rotation of the articulation mechanism relative to the second electronic drive assembly.
